(19) United States
(12) Patent Application Publication
(10) Pub. No.: US 2024/0099978 A1
(43) Pub. Date: Mar. 28, 2024

(54) Composite Nano-agent for Combating Drug-fast Bacteria and Preparation Method Thereof

(71) Applicant: Guangdong Medical University, Dongguan (CN)

(72) Inventors: Xingyan Liu, Dongguan (CN); Yining Liu, Dongguan (CN)

(21) Appl. No.: 18/530,276

(22) Filed: Dec. 6, 2023

(51) Int. Cl. A61K 9/127; A61K 33/38; A61K 47/10; A61K 47/32

(52) U.S. Cl. CPC A61K 9/1277; A61K 33/38; A61K 47/10; A61K 47/32

(57) ABSTRACT

Disclosed is a composite nano-agent for combating drug-fast bacteria and a preparation method thereof. The solution comprises several steps of operation that a silver salt is reduced into silver nanoparticles; then, phosphatidylcholine (PC) and cholesterol are mixed and added into water, and ultrasonic treatment is carried out to form liposomes; the silver nanoparticles and the liposomes are then mixed and an ultrasonic processor is used to uniformly mix a mixture by adding polyethylene glycol (PEG); an anti-bacterial experiment is carried out on the prepared reagent to determine a sterilization effect and a minimal inhibitory concentration (MIC) thereof.

### TECHNICAL FIELD

The present disclosure relates to the technical field of nano-agents, and more specifically, to a composite nano-agent for combating drug-fast bacteria and a preparation method thereof.

### BACKGROUND

With the wide use of antibiotics, the emergence and spread of resistant strains have become a worldwide public health issue. Therefore, to date, the development of new antibacterial agents and therapeutic methods has become one of the hot spots for research in the medical and biotechnological fields. In recent years, the progress in nanotechnology has provided new ideas and approaches for the treatment and prevention of infection. Highly efficient bactericidal and antiseptic composite nano-agents can be prepared with unique properties of nanomaterials, such as large surface area, sound biocompatibility, and strong controllability.

Silver nanoparticles are broad-spectrum bactericidal, liposomes can enhance the biocompatibility and stability of the silver nanoparticles, and Polyethylene Glycol (PEG) can prevent the nanoparticles from being recognized and removed in vivo. In the process of preparing the silver nanoparticles, silver ions need to be added to an aqueous solution containing Polyvinyl Alcohol (PVA) and irradiated with ultraviolet light. However, due to the fact that a penetrating power of the ultraviolet light is limited, it is difficult to effectively irradiate a deeper layer of a mixture at a higher efficiency when the mixture is irradiated.

Therefore, it is necessary to provide a composite nano-agent for combating drug-fast bacteria and a preparation method thereof to solve the above technical problem.

### SUMMARY

The objective of the present disclosure is to provide a composite nano-agent for combating drug-fast bacteria and a preparation method thereof to address the above-mentioned problem.

To achieve the above-mentioned objective, one embodiment of the present disclosure provides the technical solution as follows:

A composite nano-agent for combating drug-fast bacteria, where the composite nano-agent material comprises:
silver nanoparticles;
silver ions;
polyvinyl alcohol (PVA);
polyethylene glycol (PEG);
tetrabutylammonium hydroxide (TBAH); and
liposomes.

A preparation method of a composite nano-agent for combating drug-fast bacteria, comprising the following steps of:
S1: Preparing silver ions: Adding a silver salt into water and adding TBAH to hydrolyze the silver salt;
S2: Preparing silver nanoparticles: Adding silver ions into an aqueous solution containing PVA and reducing the silver ions into the silver nanoparticles in reduction equipment;
S3: Preparing liposomes: Mixing phosphatidylcholine (PC) and cholesterol, adding the mixture into water, and carrying out ultrasonic treatment to form the liposomes;
S4: Mixing the silver nanoparticles and the liposomes and adding PEG to stabilize the mixture;
S5: Characterizing the mixture; and
S6: Carrying out an anti-bacterial experiment on the composite nano-agent and determining a sterilization effect and a minimal inhibitory concentration (MIC) thereof.

As a further improvement of the present disclosure, the range of a ratio of the silver salt to the TBAH in the S1 is 1:1 to 1:3.

As a further improvement of the present disclosure, the range of a ratio of the silver ions to the PVA in the S2 is 1:1 to 1:10.

As a further improvement of the present disclosure, the reduction equipment in the S2 comprises a reduction tank, a segmentation assembly is arranged in the reduction tank, and a sealing cap is arranged on an upper side of the segmentation assembly.

As a further improvement of the present disclosure, the segmentation assembly comprises a transparent cylinder, base plates, a hollowed-out ring, and top plates. A plurality of top plates and a plurality of base plates which are uniformly distributed are installed outside the transparent cylinder in a surrounding manner, the hollowed-out ring is installed outside the transparent cylinder and located in middles of the top plates and the base plates, and a top end and a bottom end of the hollowed-out ring are securely connected with the top plates and the base plates, respectively.

As a further improvement of the present disclosure, an ultraviolet lamp matched with a length of the transparent cylinder is installed at a bottom end of the sealing cap and located inside the transparent cylinder.

As a further improvement of the present disclosure, the specific operation steps in the S3 comprise:
S31: Preparing PC and cholesterol;
S32: Placing an appropriate amount of PC and the cholesterol into a dry, sterile, and high-temperature-resistant glass beaker;
S33: Adding an appropriate amount of deionized water into the beaker and ensure even mixing;
S34: Placing the beaker into an ultrasonic processor and treating a mixture by using a suitable ultrasonic power and time; and
S35: Checking the quality and characteristics of the liposomes after preparation.

As a further improvement of the present disclosure, the range of a mixing ratio of the silver nanoparticles to the liposomes in the S4 is 1:1 to 1:10, and the range of the addition amount of PEG in the S4 is 5%-10% of the total mass of the silver nanoparticles and the liposomes.

As a further improvement of the present disclosure, the parameters for characterizing the mixture in the S5 include a particle size, a dispersity, a morphology, a structure and an optical property.

Compared with the prior art, the advantages of the present disclosure are presented as follows:

The solution comprises the steps of adding a silver salt into water, and adding TBAH to hydrolyze the silver salt to obtain silver ions; adding the silver ions into an aqueous solution containing PVA, reducing the silver ions into silver nanoparticles in reduction equipment, and homogeneously irradiating solutions at various layers of the mixture through the reduction equipment to improve the efficiency of reducing the silver ions into the silver nanoparticles and the efficiency of reagent preparation; then, mixing PC and cholesterol, adding the mixture into water, and carrying out ultrasonic treatment to form liposomes; mixing the silver nanoparticles and the liposomes and adding PEG to stabilize the mixture, mixing an appropriate amount of silver nanoparticles and liposomes, adding PEG to the mixture to be uniformly mixed using an ultrasonic processor; subsequently, characterizing the mixture thereof; and carrying out an anti-bacterial experiment on the prepared reagent after characterization to determine the sterilization effect and MIC thereof.

### BRIEF DESCRIPTION OF THE DRAWINGS

FIG. 1 is a schematic flowchart of a preparation method of a composite nano-agent for combating drug-fast bacteria of the present disclosure.

FIG. 2 is a schematic diagram for a three-dimensional structure of reduction equipment of the present disclosure.

FIG. 3 is a schematic diagram for a three-dimensional structure of a segmentation assembly of the present disclosure.

### DESCRIPTION OF REFERENCE SIGNS

1. Reduction tank; 2. Segmentation assembly; 3. Sealing cap; 21. Transparent cylinder; 22. Base plate; 23. Hollowed-out ring; 24. Top plate; 31. Ultraviolet lamp.

### DETAILED DESCRIPTION OF THE EMBODIMENTS

The technical solutions in the embodiment of the present disclosure will be clearly and completely described below in combination with the drawings in the embodiment of the present disclosure; obviously, the described embodiment is only a part of the embodiments of the present disclosure but not all. Based on the embodiments of the present disclosure, all the other embodiments obtained by those of ordinary skill in the art on the premise of not contributing creative effort should belong to the protection scope of the present disclosure.

### Example 1

A composite nano-agent for combating drug-fast bacteria, where the composite nano-agent material comprised:
silver nanoparticles;
silver ions;
PVA;
PEG;
TBAH; and
liposomes.

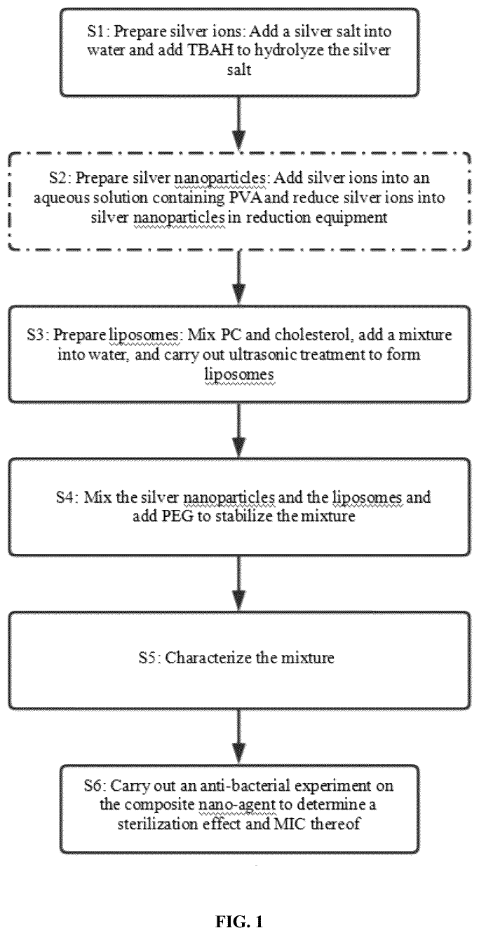

With reference to FIG. 1, a preparation method of a composite nano-agent for combating drug-fast bacteria comprised the following steps of:
S1: Preparing silver ions: Adding a silver salt into water and adding TBAH to hydrolyze the silver salt;
S2: Preparing silver nanoparticles: Adding silver ions into an aqueous solution containing PVA and reducing the silver ions into the silver nanoparticles in reduction equipment;
S3: Preparing liposomes: Mixing PC and cholesterol, adding the mixture into water, and carrying out ultrasonic treatment to form the liposomes;
S4: Mixing the silver nanoparticles and the liposomes and adding PEG to stabilize the mixture;
S5: Characterizing the mixture; and
S6: Carrying out an anti-bacterial experiment on the composite nano-agent and determining a sterilization effect and MIC thereof.

The solution comprised the steps of adding the silver salt into the water, and adding TBAH to hydrolyze the silver salt to obtain silver ions; adding the silver ions into an aqueous solution containing PVA, reducing the silver ions into silver nanoparticles in reduction equipment, and homogeneously irradiating solutions at various layers of the mixture through the reduction equipment to improve the efficiency of reducing the silver ions into the silver nanoparticles and the efficiency of reagent preparation; then, mixing PC and cholesterol, adding the mixture into water, and carrying out ultrasonic treatment to form the liposomes; mixing the silver nanoparticles and the liposomes and adding PEG to stabilize the mixture, mixing an appropriate amount of silver nanoparticles and liposomes, adding PEG to the mixture to be uniformly mixed using an ultrasonic processor; subsequently, characterizing the mixture thereof; and carrying out an anti-bacterial experiment on the prepared reagent after characterization to the its sterilization effect and MIC thereof.

The range of a ratio of the silver salt to TBAH in S1 was 1:1 to 1:3, and the range of a ratio of the silver ions to PVA in S2 was 1:1 to 1:10.

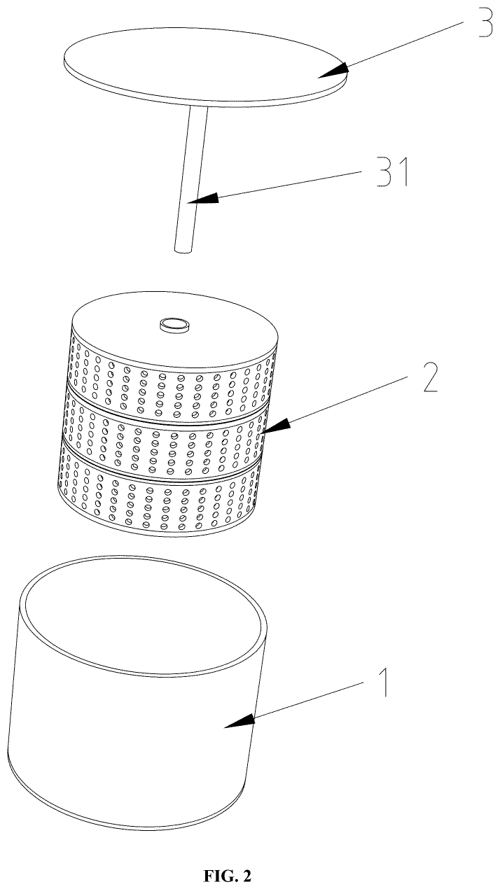

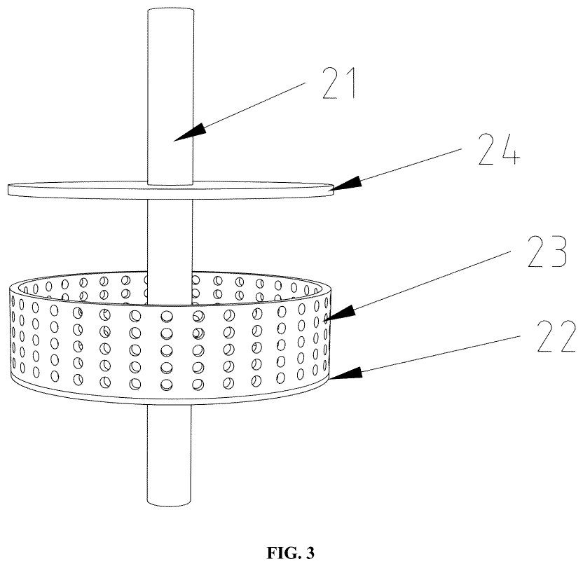

With reference to FIG. 2 and FIG. 3, the reduction equipment in S2 comprised a reduction tank 1, a segmentation assembly 2 is arranged in the reduction tank 1, and a sealing cap 3 is arranged on an upper side of the segmentation assembly 2. The segmentation assembly 2 comprised a transparent cylinder 21, base plates 22, a hollowed-out ring 23 and top plates 24. A plurality of top plates 24 and a plurality of base plates 22 which were uniformly distributed were installed outside the transparent cylinder 21 in a surrounding manner, the hollowed-out ring 23 was installed outside the transparent cylinder 21 and located in middles of the top plates 24 and the base plates 23, and a top end and a bottom end of the hollowed-out ring 23 were securely connected with the top plates 24 and the base plates 23, respectively. An ultraviolet lamp 31 matched with a length of the transparent cylinder 21 was installed at a bottom end of the sealing cap 3 and located inside the transparent cylinder 21.

The silver ions were added into an aqueous solution containing PVA, the mixture was poured into the reduction tank 1, and then the segmentation assembly 2 was slowly placed into the reduction tank 1, so that the mixture could enter the hollowed-out ring 23. The mixture could be divided into multiple relatively confined spaces through a plurality of top plates 24 and base plates 22, as well as the hollowed-out ring 23 in middles of the top plates 24 and the base plates 22. The ultraviolet lamp 31 installed on the sealing cap 3 was placed inside the transparent cylinder 21 and a power switch of the ultraviolet lamp 31 was turned on, so that the mixture in the reduction tank 1 was irradiated by the ultraviolet light emitted by the ultraviolet lamp 31, thereby ensuring that mixture at various layers could be steadily and uniformly irradiated.

Meanwhile, ends, close to each other, of the rear top plates 24 and the base plates 22 and an inner surface of the hollowed-out ring 23 were coated with titanium oxide coatings, so that the ultraviolet light could be reflected to a large extent, and the ultraviolet light emitted by the ultraviolet lamp 31 could act on the mixture to a maximum extent in a relatively confined space formed by the base plates 22, the hollowed-out ring 23 and the top plates 24, so that the efficiency of reducing the silver nanoparticles by the mixture at different depths could be accelerated, and the reagent preparation efficiency could be improved.

The specific operation steps in S3 comprised:
S31: Preparing PC and cholesterol;
S32: Placing an appropriate amount of PC and the cholesterol into a dry, sterile, and high-temperature-resistant glass beaker;
S33: Adding an appropriate amount of deionized water into the beaker and ensure even mixing;
S34: Placing the beaker into an ultrasonic processor and treating the mixture by using a suitable ultrasonic power and time; and
S35: Checking the quality and characteristics of the liposomes after preparation.

Preparing PC and cholesterol meant preparing PC and cholesterol in the required proportion and quantity; placing a suitable amount of PC and cholesterol into a dry, sterile, and high-temperature-resistant glass beaker meant placing the mixture of the prepared PC and cholesterol into a glass beaker; adding an appropriate amount of deionized water into the beaker and ensuring the even mixing meant adding the deionized water into the beaker and mixing evenly with a glass stick to ensure that PC and cholesterol were evenly dispersed in the water; placing the beaker into an ultrasonic processor and treating the mixture by using the suitable ultrasonic power and time meant placing the beaker into an ultrasonic processor, setting appropriate ultrasonic power and time, and processing the mixture to form the liposomes; and checking the quality and characteristics of the liposomes after preparation meant that after the liposomes were prepared, the quality and characteristics of the liposomes were checked to ensure that the liposomes met the required quality standards and characteristic requirements.

The range of a ratio of the silver nanoparticles to the liposomes in S4 was 1:1 to 1:10, and the addition amount of PEG in S4 was 5% to 10% of the total mass of the silver nanoparticles and the liposomes.

The parameters for characterizing the mixture in S5 included a particle size, a dispersity, a morphology, a structure and an optical property.

The particle size of the nanoparticles is an important parameter. A dynamic light scatterometer can measure an average particle size, a distribution condition, an aggregation state and other information of the nanoparticles, and a transmission electron microscope (TEM) can be used to directly observe the morphology and size of the nanoparticles.

The dispersity of nanoparticles refers to the uniformity of the nanoparticles in the solution; an ultraviolet-visible (UV-Vis) spectroscopy can measure the absorption spectrum of the nanoparticles, analyze the dispersity and aggregation state of the nanoparticles, and a Zeta potential analyzer can measure a state of electric charge on the surfaces of the nanoparticles, thereby determining the stability and dispersity of the nanoparticles.

The morphology and structure of nanoparticles can be observed and analyzed by a high-resolution microscope such as a TEM.

The optical property of the nanoparticles can be characterized by an UV-vis spectroscopy or a surface-enhanced Raman spectroscopy (SERS) method.

It will be apparent to those skilled in the art that the present disclosure is not limited to the details of the above exemplary embodiment, and those skilled in the art may implement the present disclosure in other specific forms without departing from the spirit and basic features of the present disclosure. Therefore, in any case, the embodiment should be regarded as the exemplary embodiment, rather than the restrictive embodiment. The scope of the present disclosure is limited by the appended claims instead of the above description. Therefore, all changes in the meaning and scope of the equal conditions of the claims shall be included in the present disclosure. Any reference sign in the claims shall not be regarded as a limitation to the involved claims.

In addition, it should be understood that, although the specification is described according to an embodiment, not each embodiment only includes one independent technical solution. The narration mode of the specification is merely for the sake of clarity, and those skilled in the art should regard the specification as a whole, and the technical solutions in the embodiments may also form other implementations, understood by those skilled in the art, in a suitable combination.

What is claimed is:

1. A composite nano-agent for combating drug-fast bacteria, wherein the composite nano-agent material comprises:
silver nanoparticles;
silver ions;
polyvinyl alcohol (PVA);
polyethylene glycol (PEG);
tetrabutylammonium hydroxide (TBAH); and
liposomes.

2. A preparation method of a composite nano-agent for combating drug-fast bacteria, wherein the method comprises the following steps of:
S1: Preparing silver ions: Adding a silver salt into water and adding TBAH to hydrolyze the silver salt;
S2: Preparing silver nanoparticles: Adding silver ions into an aqueous solution containing PVA and reducing the silver ions into the silver nanoparticles in reduction equipment;
S3: Preparing liposomes: Mixing phosphatidylcholine (PC) and cholesterol, adding the mixture into water, and carrying out ultrasonic treatment to form the liposomes;
S4: Mixing the silver nanoparticles and the liposomes and adding PEG to stabilize the mixture;
S5: Characterizing the mixture; and
S6: Carrying out an anti-bacterial experiment on the composite nano-agent and determining a sterilization effect and MIC thereof.

3. The preparation method of the composite nano-agent for combating the drug-fast bacteria according to claim 2, wherein the range of a ratio of the silver salt to TBAH in the S1 is 1:1 to 1:3.

4. The preparation method of the composite nano-agent for combating the drug-fast bacteria according to claim 2, wherein the range of a ratio of the silver ions to PVA in the S2 is 1:1 to 1:10.

5. The preparation method of the composite nano-agent for combating the drug-fast bacteria according to claim 2, wherein the reduction equipment in the S2 comprises a reduction tank (1), a segmentation assembly (2) arranged in the reduction tank (1), and a sealing cap (3) is arranged on an upper side of the segmentation assembly (2).

6. The preparation method of the composite nano-agent for combating the drug-fast bacteria according to claim 5, wherein the segmentation assembly (2) comprises a transparent cylinder (21), base plates (22), a hollowed-out ring (23), and top plates (24); a plurality of top plates (24) and a plurality of base plates (22) which are uniformly distributed are installed outside the transparent cylinder (21) in a surrounding manner, and the hollowed-out ring (23) is installed outside the transparent cylinder (21) and located in middles of the top plates (24) and the base plates (22), and a top end and a bottom end of the hollowed-out ring (23) are securely connected with the top plates (24) and the base plates (22), respectively.

7. The preparation method of the composite nano-agent for combating the drug-fast bacteria according to claim 5, wherein an ultraviolet lamp (31) matched with a length of the transparent cylinder (21) is installed at a bottom end of the sealing cap (3) and located inside the transparent cylinder (21).

8. The preparation method of the composite nano-agent for combating the drug-fast bacteria according to claim 2, wherein the specific operation steps in the S3 comprise:
S31: Preparing PC and cholesterol;
S32: Placing an appropriate amount of PC and the cholesterol into a dry, sterile, and high-temperature-resistant glass beaker;
S33: Adding an appropriate amount of deionized water into the beaker and ensure even mixing;
S34: Placing the beaker into an ultrasonic processor and treating a mixture by using a suitable ultrasonic power and time; and
S35: Checking the quality and characteristics of the liposomes after preparation.

9. The preparation method of the composite nano-agent for combating the drug-fast bacteria according to claim 2, wherein the range of a mixing ratio of the silver nanoparticles to the liposomes in the S4 is 1:1 to 1:10, and the addition amount of PEG in the S4 is 5% to 10% of the total mass of the silver nanoparticles and the liposomes.

10. The preparation method of the composite nano-agent for combating the drug-fast bacteria according to claim 2, wherein the parameters for characterizing the mixture in the S5 comprises a particle size, a dispersity, a morphology, a structure, and an optical property.
